(19) United States
(12) Patent Application Publication
(10) Pub. No.: US 2009/0196865 A1
(43) Pub. Date: Aug. 6, 2009

(54) Methods for the treatment and prevention of diseases of biological conduits

(75) Inventor: F. Nicholas Franano, Kansas City, MO (US)

Correspondence Address: DECHERT LLP, P.O. BOX 390460, MOUNTAIN VIEW, CA, 94039-0460, US

(73) Assignee: PROTEON THERAPEUTICS, INC., Kansas City, MO (US)

(21) Appl. No.: 11/663,615

(22) PCT Filed: Sep. 22, 2005
(86) PCT No.: PCT/US05/34200

§ 371 Date: Jun. 19, 2008

Related U.S. Application Data
(60) Provisional application No. 60/612,296, filed on Sep. 22, 2004.

(51) Int. Cl. A61K 38/48; A61P 9/14

(52) U.S. Cl. 424/94.64

(57) ABSTRACT

Methods for treating or preventing disease in biological conduits are provided herein. In certain embodiments, the methods relate to reducing or preventing vasospasm in blood vessel walls. In other embodiments, the methods described herein relate to reducing the accumulation of intimal hyperplasia in blood vessel walls after vascular procedures, including surgery. The methods encompass the use of agents that are useful for dilating biological conduits, but in dosages lower than are effective to achieve dilation of biological conduits.

This application claims the benefit of U.S. Provisional Application No. 60/612,296, filed on Sep. 22, 2004, which is incorporated by reference herein in its entirety.

### FIELD OF THE INVENTION

The present invention relates to methods for treating or preventing disease in biological conduits. In certain embodiments, the methods described herein relate to reducing or preventing vasospasm in blood vessel walls. In other embodiments, the methods described herein relate to reducing the accumulation of intimal hyperplasia in blood vessel walls after vascular procedures, including surgery.

### BACKGROUND OF THE INVENTION

### 2.1. Blood Vessel Structure

A blood vessel is composed of three distinct layers. From inside to outside, these layers include the intima, the media and the adventitia. The intima is usually comprised of a single layer of flat endothelial cells that line the lumen of the vessel. The medial layer is composed of sheets of smooth muscle cells and extracellular matrix fibers. The adventitia is an outer layer that comprises a covering of extracellular matrix and scattered fibroblasts. The extracellular matrix of blood is organized around a weave of two protein fibers, one composed predominantly of elastin and the other of collagen. The elastin fiber can be extended to nearly twice its initial length and still recoil completely. Under normal hemodynamic conditions, elastin fibers are taut and exert a retractive force on the wall of the vessel that counters the force of distension created by the pumping of the heart. In contrast, the collagen fiber is relatively rigid, but at normal vessel diameters, collagen fibers are slack and contribute little to wall tension. Vascular smooth muscle cells are connected to the elastin fiber network through a series of junctions. When a vessel is injured, the vascular smooth muscle cells can contract in a coordinated fashion, resulting in a constriction of the injured vessel segment and a reduction of flow to the damaged segment of vessel, a process known as vasospasm. Vasospasm is a protective response to trauma, and can act to limit bleeding. During and after vascular surgery procedures however, vasospasm is usually undesirable and can increase the risk of ischemia in the tissues fed by the vessel, and thrombosis of the affected vessel segment, leading to infarction. Vascular injury can also lead to the accumulation of vascular smooth muscle cells in the intimal layer, a pathologic lesion known as “intimal hyperplasia”. This buildup of cells and associated extracellular matrix leads to gradual lumen narrowing and if severe enough vessel obstruction. Vessels are injured during surgery by the cutting and suturing process. Vessels are also injured during angioplasty by the stretching, tearing, and wall compression caused by balloon inflation. Vessels can also be injured by chronic, abnormal hemodynamic forces such as those caused by compliance mismatch at vessel anastomosis, or when veins are placed in a high pressure or pulsatile environment as with bypass grafting or hemodialysis access site creation.

### 2.2. Vasospasm and Intimal Hyperplasia in Bypass Grafts

Large arteries can become completely obstructed by the accumulation of atherosclerotic plaque in the wall of a segment of the artery, leading to ischemia and infarction of the downstream tissues. This obstruction is often treated with bypass grafting, where the blocked segment is bypassed using either autologous vein or artery, or a conduit made of synthetic materials such as Dacron or polytetrafluoroethylene (“PTFE”). During this procedure, the distal end of the bypass conduit is connected to an artery beyond the obstruction, thereby diverting the flow of blood around the obstructed segment, and providing adequate flow to the downstream tissues. When small vessels are used in the construction of a bypass graft, there is increased risk for early graft failure and thrombosis. Injury to the vessels during bypass graft surgery can result in vasospasm, both during the surgery and in the post-operative period, leading to a further reduction in lumen diameter and an increased risk of ischemia and thrombosis. Injury to vessels during and after bypass grafting also leads to an accumulation of vascular smooth muscle cells in the intimal layer where they form a lesion known as intimal hyperplasia that reduces the diameter of the vessel lumen. There is a need for strategies to reduce the risk of vasospasm during and after the creation of bypass grafts and to reduce the accumulation of intimal hyperplasia in vessel walls after bypass graft surgery.

### 2.3. Vasospasm and Intimal Hyperplasia in Hemodialysis Access Sites

Patients whose kidneys no longer function adequately undergo periodic external blood filtering, a process known as hemodialysis. To prepare for hemodialysis, vascular surgeons create high flow access sites in the body that can easily be connected to hemodialysis machines. High flow rates are created in the sites by connecting an artery to a vein, resulting in a “shunt” of blood through the vessels. Permanent hemodialysis access sites come in two forms: AV fistulas and AV grafts.

An AV fistula is constructed by creating a direct connection between an artery and vein. The vein leading away from the connection is called the “outflow vein”. This vein usually dilates naturally by 25-40% over a period of 1-3 months and the vein becomes visible under the skin, a process known as “maturation”. Upon maturation, the outflow vein can be easily accessed with needles for hemodialysis. A functional AV fistula is the most durable, longest-lasting form of hemodialysis access, with a mean patency of 3 years. However, a large fraction of newly created AV fistulas can never be used, often because of thrombosis in the post-operative period, an event that is often caused by vasospasm. An AV graft is constructed by placing a synthetic conduit between an artery and vein. A portion of the conduit is placed immediately under the skin for easy hemodialysis access. AV grafts also fail in the post-operative period because of vasospasm and thrombosis, although less frequently than AV fistulas. The main risk factor for early failure of AV fistulas and grafts is small artery and vein diameter. This factor can be exacerbated by vasospasm, leading to a further reduction in lumen diameter and an increased risk of acute thrombosis of the site. Another factor that can reduce lumen diameter after surgery is the accumulation of vascular smooth muscle cells in the intimal layer where they form a lesion known as intimal hyperplasia that reduces the diameter of the vessel lumen. Intimal hyperplasia usually appears to the greatest degree in the outflow vein of hemodialysis access sites. There is a need for strategies to reduce the risk of vasospasm during and after the creation of AV fistulas and grafts, and also strategies to reduce the accumulation of intimal hyperplasia in the wall of vessels used to create these sites.

### 2.4. Vasospasm During Angioplasty

When large arteries are severely narrowed, but not completely occluded, a high-pressure balloon can be inserted into the narrowed segment and inflated in order to enlarge the lumen of the narrowed segment, a procedure known as balloon angioplasty. The mechanical stimulus of the wall of the vessel during angioplasty can cause vasospasm in the treated vessel, resulting in decreased lumen diameter and flow, and increasing the risk of acute vessel thrombosis. In the absence of vasospasm, the balloon enlarges the lumen, often by tearing the wall and disrupting the network of collagen and elastin fibers. The tearing of the arterial wall is associated with mural thrombus formation, platelet deposition, and subsequent narrowing of the lumen at the treatment site by organizing mural thrombus and the accumulation of vascular smooth muscle cells in the intimal layer. There is a need for strategies to reduce the risk of vasospasm during and after balloon angioplasty and also strategies to reduce the accumulation of intimal hyperplasia in the wall of vessels treated with balloon angioplasty.

Citation or identification of any reference in Section 2 or in any other section of this application shall not be construed as an admission that such reference is available as prior art to the present invention.

### SUMMARY OF THE INVENTION

The present invention provides methods of treating or preventing vasospasm in a biological conduit, and for reducing the accumulation of intimal hyperplasia in a biological conduit after a vascular procedure, by administering an elastase, a collagenase, or an agent that increases the local concentration of an elastase or collagenase, wherein the effective amount for such treatment or prevention is surprisingly smaller than the amounts previously found to be effective for dilating the biological conduit.

The application of high doses of elastases to the wall of arteries and veins can cause persistent vasodilation in the treated segment, reduced vasospasm, and under certain circumstances, a reduction in the accumulation of intimal hyperplasia after treatment. These effects can decrease the risk of obstruction after vascular treatments, in part by increasing the capacity of the treated segments to transmit blood. At lower elastase doses, no obvious persistent vessel dilation is observed. However, at these lower doses of elastase, vasospasm and/or the accumulation of intimal hyperplasia after treatment can also be reduced.

Accordingly, the present invention provides methods of treating or preventing disease in a biological conduit. In certain aspects, the invention provides methods of treating vessels with elastases in order to reduce vasospasm and/or reduce the ability of the treated vessel segment to undergo vasospasm. The present inventor has demonstrated that arteries treated with elastase are resistant to vasospasm-inducing pharmacologic agents and trauma. The resistance of elastase-treated vessel segments to vasospasm provides added protection against ischemia and thrombosis after vascular procedures. In another aspect, the invention provides methods to reduce the accumulation of intimal hyperplasia in the wall of vessels after vascular surgery procedures. The present inventor has demonstrated that outflow veins from hemodialysis access sites that are treated with elastases develop much less intimal hyperplasia over time, when compared with sites receiving control treatments. The resistance of elastase-treated vessel segments to vasospasm and the reduction in the accumulation of intimal hyperplasia provides added protection against ischemia and thrombosis after vascular procedures.

In certain aspects, the invention provides methods of treating or preventing disease in a biological conduit by one or more of the following, in any desired combination, (a) administering one or more exogenous elastases to the conduit or to a wall of the conduit; (b) administering one or more exogenous collagenases to the conduit or to a wall of the conduit; (c) increasing the local concentration of one or more endogenous elastases and/or collagenases in the conduit or in a wall of the conduit; (d) inducing inflammation locally in the conduit or in a wall of the conduit; (e) degrading microfibers locally in the conduit or in a wall of the conduit; (f) increasing the local concentration of an endogenous chemotactic factor for monocytes, macrophages, or polymorphonuclear cells in the conduit or in a wall of the conduit; (g) activating macrophages in the conduit or in a wall of the conduit; (h) degrading extracellular matrix in the conduit or in a wall of the conduit; and/or (i) degrading proteoglycans or glycoproteins in the conduit or in a wall of the conduit. The agents administered, are in amounts that, cumulatively, are insufficient to dilate the biological conduit but sufficient to reduce vasospasm and/or reduce the ability of the treated vessel segment to undergo vasospasm and/or reduce the accumulation of intimal hyperplasia in the wall of vessels after a vascular surgery procedure.

As used herein, the term “endogenous” means produced by the subject being treated in accordance with the methods of the invention. As used herein, the term “exogenous” means produced by a source other than the subject being treated in accordance with the methods of the invention.

In certain specific embodiments, a single agent is utilized that can achieve one or more effects enumerated in (a)-(i) above such that vasospasm and/or the ability of the treated vessel segment to undergo vasospasm and/or the accumulation of intimal hyperplasia in the wall of vessels after a vascular surgery procedure is reduced, without at the same time dilating the biological conduit. In other embodiments, combination therapy entailing the administration of one or more agents may be used to achieve one or more of the effects enumerated in (a)-(i) above such that such that vasospasm and/or the ability of the treated vessel segment to undergo vasospasm and/or the accumulation of intimal hyperplasia in the wall of vessels after a vascular surgery procedure is reduced, without at the same time dilating the biological conduit.

Exemplary agents that may achieve one or more effects include, for example, matrix metalloproteinase-1, whose substrates include native collagen types III, I, II, VII, X, aggrecan, link protein, entactin, tenascin, and perlecan; matrix metalloproteinase-6, whose substrates include native collagen types I, II, III, VII, X, aggrecan, entactin, and tenascin matrix metalloproteinase-13, whose substrates include native collagen types II, III, I, VII, X, aggrecan, entactin, and tenascin; matrix metalloproteinase-18, whose substrates include native collagen types I, II, and III; matrix metalloproteinase-14, whose substrates include native collagen types I, II, III, aggrecan, fibronectin, and vitronectin; matrix metalloproteinase-16, whose substrates include native type III collagen and fibronectin; matrix metalloproteinase-24, whose substrates include fibronectin and proteoglycans; matrix metalloproteinase-25, whose substrates include native type IV collagen, fibronectin, proteoglycans (DSPG, CSPG), laminin-1, and fibrin/fibrinogen; matrix metalloproteinase-2, whose substrates include elastin, native collagen types I, IV, V, VII, X, XI; matrix metalloproteinase-9, whose substrates include elastin, native collagen types I, IV, V, VII, X, and XI, fibronectin, laminin, aggrecan, link protein and vitronectin.

In certain embodiments, the cumulative dosage of the agent or agents employed in the methods of the invention is preferably at least 5%, 10%, 15%, 20%, 25%, 30%, 35%, 40%, 50%, 60%, 70%, 80% or 90% less than that dosage required to achieve dilation of the biological conduit.

In certain aspects, the present invention provides methods of treating or preventing vasospasm in at least a segment of a biological conduit and/or for reducing the accumulation of intimal hyperplasia in at least a segment of a biological conduit after a vascular procedure, said methods comprising increasing the local concentration of one or more endogenous elastases or collagenases, wherein said increase is not achieved by administration of an elastase or collagenase.

In other aspects, the present invention provides methods of treating or preventing vasospasm in at least a segment of a biological conduit and/or for reducing the accumulation of intimal hyperplasia in at least a segment of a biological conduit after a vascular procedure, said methods comprising increasing the local concentration of one or more endogenous elastases and/or collagenases and/or administering one or more exogenous elastases and/or collagenases to the conduit or to a wall of the conduit.

In other aspects, the present invention provides methods of treating or preventing vasospasm in at least a segment of a biological conduit and/or for reducing the accumulation of intimal hyperplasia in at least a segment of a biological conduit after a vascular procedure, said methods comprising inducing local inflammation in said segment.

In yet other aspects, the present invention provides methods of treating or preventing vasospasm in at least a segment of a biological conduit and/or for reducing the accumulation of intimal hyperplasia in at least a segment of a biological conduit after a vascular procedure, said methods comprising increasing the local concentration of one or more endogenous elastases and/or collagenases and/or administering one or more exogenous elastases and/or collagenases to the segment or to a wall of the segment and inducing local inflammation in said segment.

In yet other aspects, the present invention provides methods of treating or preventing vasospasm in at least a segment of a biological conduit and/or for reducing the accumulation of intimal hyperplasia in at least a segment of a biological conduit after a vascular procedure, said methods comprising increasing the local concentration of one or more endogenous elastases and/or collagenases and/or administering one or more exogenous elastases and/or collagenases to the segment or to a wall of the segment, and degrading microfibers in the wall of said segment.

In yet other aspects, the present invention provides methods of treating or preventing vasospasm in at least a segment of a biological conduit and/or for reducing the accumulation of intimal hyperplasia in at least a segment of a biological conduit after a vascular procedure, said methods comprising increasing the local concentration of one or more endogenous chemotactic factors for monocytes, macrophages, or polymorphonuclear cells and/or administering one or more exogenous chemotactic factors for monocytes, macrophages, or polymorphonuclear cells to the segment or to a wall of the segment, and activating macrophages locally, for example by increasing the local concentration of one or more endogenous macrophage-activating agents and/or administering one or more exogenous macrophage-activating agents to said segment or to a wall of the segment.

In yet other aspects, the present invention provides methods of treating or preventing vasospasm in at least a segment of a biological conduit and/or for reducing the accumulation of intimal hyperplasia in at least a segment of a biological conduit after a vascular procedure, said methods comprising (i) administering one or more exogenous elastases and/or collagenases to the conduit or to a wall of the conduit and/or increasing the local concentration of one or more endogenous elastases and/or collagenases; (ii) administering one or more exogenous chemotactic factors for monocytes, macrophages, or polymorphonuclear cells to the conduit or to a wall of the conduit and/or increasing the local concentration of one or more chemotactic factors for monocytes, macrophages, or polymorphonuclear cells; and (iii) activating macrophages locally, for example by increasing the local concentration of one or more endogenous macrophage-activating agents and/or administering one or more exogenous macrophage-activating agents to the conduit or to a wall of the conduit.

In yet other aspects, the present invention provides methods of treating or preventing vasospasm in at least a segment of a biological conduit and/or for reducing the accumulation of intimal hyperplasia in at least a segment of a biological conduit after a vascular procedure, and at the same time inhibiting enlargement of at least a segment of a biological conduit, wherein enlargement of at least a segment of a biological conduit is inhibited by antagonizing a PAR receptor.

Another aspect of the present invention provides methods of treating or preventing vasospasm in at least a segment of a biological conduit and/or for reducing the accumulation of intimal hyperplasia in at least a segment of a biological conduit after a vascular procedure, comprising administering to the wall of the conduit an agent that induces local inflammation and/or results in the recruitment of monocytes, macrophages, and/or polymorphonuclear (PMN) cells capable of synthesizing and releasing elastases and collagenases in the conduit wall. In some embodiments, the administered agent would be chemotactic for these cells. In one embodiment, one or more of the chemotactic agents comprises of monocyte chemotactic peptide-1, granulocyte macrophage colony stimulating factor, tumor necrosis factor alpha, or an interleukin. In other embodiments, the agent would cause the local synthesis and/or release of endogenous agents that are chemotactic for monocytes, macrophages, or PMNs. One or more of said chemotactic agents comprises monocyte chemotactic peptide-1, granulocyte macrophage colony stimulating factor, tumor necrosis factor alpha, interferon gamma, leukotriene B4, C5a, interleukin-1, or interleukin-8.

In certain embodiments, the agents employed in the methods of the invention are capable of acting synergistically.

The present invention provides methods of treating or preventing vasospasm in at least a segment of a biological conduit and/or for reducing the accumulation of intimal hyperplasia in at least a segment of a biological conduit after a vascular procedure by administering to the biological conduit a first composition comprising one or more chemotactic factors for monocytes, macrophages, or polymorphonuclear cells and a second composition comprising an agent that is a macrophage-activating agent. In one embodiment, one or more of the chemotactic agents is not an elastase or a collagenase. In another embodiment, the macrophage-activating agent is a bacterial lipopolysaccharide, thioglycollate, or CpG DNA. In a further embodiment, the first and second compositions are the same and/or are administered in synergistic amounts. In a further embodiment, the first composition and second composition are administered concurrently or the first composition is administered prior to the second composition or the second composition is administered prior to the first composition. In an embodiment, the biological conduit is an artery or vein, or an arterial or venous vascular graft.

In the present invention, the administered agent can activate one or more members of the G-protein coupled proteinase activated receptor (PAR) family. Four distinct PARs are known, and they have been given the names PAR-1 (thrombin receptor), PAR-2, PAR-3, and PAR-4. PARs are activated when an n-terminal peptide is cleaved from the receptor, revealing a tethered ligand that inserts into the receptor-binding site. PAR receptor activation often leads to tissue inflammation and the recruitment of monocytes, macrophages, and PMNs. In some embodiments, the agent causes increased expression of the endogenous PAR receptor in the target tissue. Preferably, the administered agent is selected from trypsin, trypsin IV, chymotrypsin, mesotrypsin, mast cell trypstase, neutrophil proteinase-1, tissue factor, factor VIIa, factor Xa, thrombin, plasmin, cathepsin G, MCP-1, a PAR-activating peptide, a PAR-activating peptidomimetic, and all members of the family of proteases known as matrix metalloproteinase (Cottrell et al., 2004, J Biol Chem. Jan. 15, 2004 [Epub ahead of print]). Alternatively, agents that induce expression of endogenous PAR-2 such as TNF-alpha, IL-1 or bacterial Lipopolysaccharide (LPS) are used (Nystedt et al., J. Biol. Chem. 271:14910).

The present invention provides methods of treating or preventing vasospasm in at least a segment of a biological conduit and/or for reducing the accumulation of intimal hyperplasia in at least a segment of a biological conduit after a vascular procedure by administering to the biological conduit a first composition comprising an elastase and, optionally, a second composition comprising an agent that degrades microfibers in the wall of the biological conduit. In one embodiment, the agent degrades one or more fibrillin components of the microfibers. In a further embodiment, the elastase is of type I or type II. In a further embodiment, the first composition comprising an elastase is a pancreatic elastase, macrophage elastase, leukocyte elastase, or a matrix metalloproteinase.

An aspect of the present invention provides a method of enhancing the efficacy of a first agent at a biological conduit by administering to the biological conduit a first composition comprising first agent and a second composition comprising a second agent that degrades one or more glycoproteins or proteoglycans in the wall of the biological conduit in order to increase the permeability of the wall of the biological conduit to the first agent. In one embodiment, the first agent is an elastase or collagenase wherein, the administration is effective to increase the external and/or luminal diameter of the biological conduit. In one embodiment, the first agent is an anti-restenosis agent. In a further embodiment, the first agent is a population of cells wherein, the cells are cardiac myocytes or stem or progenitor cells capable of differentiating into cardiac myocytes, and wherein the first and second compositions are administered percutaneously into the adventitial space. In a further embodiment, the first and second compositions are the same and/or are administered in synergistic amounts. In a further embodiment, the first composition and second composition are administered concurrently or the first composition is administered prior to the second composition or the second composition is administered prior to the first composition. In an embodiment, the biological conduit is an artery or vein, or an arterial or venous vascular graft.

Another aspect of the present invention involves the addition of an agent that degrades microfibers and/or fibrillins to an agent that degrades tropoelastin, for the purpose of decreasing the resynthesis of elastin fibers. Preferably, the administered agent is selected from trypsin, chymotrypsin, and plasmin, and all members of the family of proteases known as matrix metalloproteinases.

In some embodiments, the agent is administered directly to a segment of the artery or vein or vascular graft. In other embodiments, the agent is delivered into the lumen of the artery or vein or vascular graft. In some embodiments, the agent is applied to the external and/or luminal surface of the artery or vein or the vascular graft. In other embodiments, the agent is administered percutaneously into a tissue comprising the biological conduit wherein, the biological conduit is a coronary artery or vein bypass graft connected to a coronary artery. In a further embodiment, the agent is administered percutaneously to the pericardial space.

In some embodiments of the present invention, the agent causes an increase in the endothelial cell surface expression of adhesion molecules or integrins for monocytes, macrophages, and/or PMNs, including intercellular adhesion molecules (ICAMs), vascular cell adhesion molecules (VCAMs), selectins, and/or the beta 2 integrin Mac-1.

Another aspect of the present invention provides methods of treating or preventing vasospasm in at least a segment of a biological conduit and/or for reducing the accumulation of intimal hyperplasia in at least a segment of a biological conduit after a vascular procedure, comprising administering to the wall of the conduit an agent that degrades proteoglycans, in order to facilitate the delivery of macromolecules, cells, or vehicles for drug delivery (e.g., polymer microspheres) into the wall and/or surrounding tissues. Examples of proteoglycans include, but are not limited to, chondroitin sulfate, keratan sulfate, heparin sulfate, perlecan, versican, syndecan, and serglycin. Preferably, the administered agent is selected from, trypsin, chymotrypsin, and plasmin. Another aspect of the present invention provides methods of treating or preventing vasospasm in at least a segment of a biological conduit and/or for reducing the accumulation of intimal hyperplasia in at least a segment of a biological conduit after a vascular procedure, comprising administering to the wall of the conduit an agent that degrades proteoglycans and glycoproteins, in order to facilitate the degradation of elastin. Examples of glycoproteins include fibrillin-1, fibrillin-2, laminin, and fibronectin. Examples of proteoglycans are given above. Preferably, the administered agent is selected from trypsin, chymotrypsin, and plasmin, and all members of the family of proteases known as matrix metalloproteinases.

In some embodiments of the present invention, a delivery apparatus such as, for example, a catheter, a syringe, and any other types of delivery apparatus conventionally used can administer the agent in accordance with the methods of the invention. In some embodiments, administration of the agent comprises localizing a delivery apparatus in close proximity to the segment of the biological conduit to be treated. In some embodiments, during delivery of the agent by a delivery apparatus, a portion of the delivery apparatus can be inserted into the wall of the biological conduit. In some embodiments, the lumen of the biological conduit can be pressurized while the agent is delivered to the pressurized segment of the biological conduit. In some embodiments, the lumen of the biological conduit is pressurized by mechanical action. In some embodiments, the lumen of the biological conduit is pressurized with a balloon catheter. In some embodiments, the agent is administered and the pressurizing is performed by the same device. In some embodiments, the agent is administered directly to the biological conduit. In some embodiments, the biological conduit is surgically exposed and the agent is delivered into the lumen or is applied to the external surface of the biological conduit in vivo. In embodiments involving luminal delivery, blood flow through the vessel may be stopped with a clamp to allow the agent to contact the endothelium surface for longer time periods and to prevent inhibition of the agent by serum. In some embodiments, the biological conduit is surgically removed and the agent is delivered to the luminal surface and/or to the external surface of the conduit in vitro. In alternative embodiments, the agent may be delivered through a polymer formulation that is placed as a stent within the vessel to be treated, a clamp or wrap on or around the vessel to be treated, or other device in, around or near the vessel to be treated. In other embodiments, agents are percutaneously injected into a tissue region for purpose of dilating arteries and/or vein within that region. In embodiments aimed at treatment of heart vessels, agents can be delivered through an intravascular catheter, percutaneously delivered to the pericardial space, or directly applied to surgically exposed coronary vessels.

An aspect of the present invention involves the blockage of PAR receptors and signal transduction pathway(s) to treat or prevent vasospasm in at least a segment of a biological conduit and/or to reduce the accumulation of intimal hyperplasia in at least a segment of a biological conduit after a vascular procedure. The administration of a PAR antagonist may block PAR activation of cells that normally reside in the wall of the vessel (including PAR-1, PAR-2, PAR-3, and PAR-4 activation by thrombin, plasmin, and factor Xa (among others). Preferably, the administered agent is selected from either monoclonal antibodies, peptides, peptidomimetic compounds or small molecules (compounds). Alternatively, inhibitors of the PAR signal transduction pathways such as nitric oxide synthase inhibitors, PDGF, TNF-alpha and bFGF receptor antagonists, or MAPK kinase inhibitors can also be administered. Preferably, such agents would be administered orally or by intravenous or intramuscular injection.

In accordance with the present invention, the biological conduits can include, for example, an artery, vein, an arterial or venous vascular graft, ureter, bronchus, bile duct, or pancreatic duct. Further, the obstruction of the biological conduit can include, for example, a stenosis, stricture, lesion, or occlusion.

### DETAILED DESCRIPTION OF THE INVENTION

The present invention provides methods for treating or preventing disease in biological conduits and/or for delivering therapeutic and prophylactic agents to biological conduits.

The invention is based, in part, on a newly discovered dosage regimen for administration of compositions according to the methods described herein, for example compositions comprising an elastase, a collagenase, and/or an agent that increases the local concentration of an elastase or collagenase in a biological conduit.

In certain aspects, the methods of the invention entail one or more of the following, in any desired combination, (a) administering one or more exogenous elastases to the conduit or to a wall of the conduit; (b) administering one or more exogenous collagenases to the conduit or to a wall of the conduit; (c) increasing the local concentration of one or more endogenous elastases and/or collagenases in the conduit or in a wall of the conduit; (d) inducing inflammation locally in the conduit or in a wall of the conduit; (e) degrading microfibers locally in the conduit or in a wall of the conduit; (f) increasing the local concentration of an endogenous chemotactic factor for monocytes, macrophages, or polymorphonuclear cells in the conduit or in a wall of the conduit; (g) activating macrophages in the conduit or in a wall of the conduit; (h) degrading extracellular matrix in the conduit or in a wall of the conduit; and/or (i) degrading proteoglycans or glycoproteins in the conduit or in a wall of the conduit.

The present inventor has discovered that a dosage of agent used in one or more of (a)-(i) above required that is useful for treating or preventing vasospasm in a biological conduit, and for reducing the accumulation of intimal hyperplasia in a biological conduit after a vascular procedure is substantially smaller than that seen to be effective to dilate the biological conduit. In various embodiments, the dosage of agent is at least 10%, 15%, 20%, 20%, 25%, 30%, 35%, 40%, 50%, 60%, 70%, 80% or 90% smaller than the dosage required to dilate the biological conduit. In specific embodiments, the dosage is approximately 10-20%, 20-30%, 30-40%, 40-50%, 50-60%, 60-70%, 70-80%, 80-90%, 10-90%, 20-80%, 30-70%, or 40-60% less than the dosage required to dilate the biological conduit.

The present invention provides methods for reducing vasospasm in a biological conduit, including but not limited to vasospasm in response to mechanical or pharmacologic stimuli, by approximately 1% to 5%, by 5% to 25%, by 25% to 50%, or by 50% to 100%.

The present invention provides methods for reducing the accumulation of intimal hyperplasia in a biological conduit after a vascular procedure by approximately 1% to 5%, by 5% to 25%, by 25% to 50%, or by 50% to 100%. This reduction may be assessed after 1 month, 3 months, 6 months, or 1 year following the treatment.

In accordance with the present invention, treatment of the biological conduits with the agents is controlled. It has been found that while the agents are potentially beneficial in certain clinical situations, they can have untoward effects. For example, high doses of porcine pancreatic serine proteases including elastase, trypsin, and chymotrypsin (as well as other unspecified porcine proteins) can lead to severe aneurysmal dilation of arteries, and even rupture. Thus, small dosages of agents used in connection with the methods of the present invention provides controlled conditions for treating or preventing vasospasm in a biological conduit, and for reducing the accumulation of intimal hyperplasia in a biological conduit after a vascular procedure, while avoiding the potentially adverse side-effects that may be seen with high doses.

The patients on whom the methods of the invention are practiced include, but are not limited to, animals such as cows, pigs, horses, chickens, cats, dogs, etc., and are preferably mammals, and most preferably human.

The biological conduits that may be treated in accordance with the methods of the invention can include, for example, arteries, veins, ureters, bronchi, bile ducts, or pancreatic ducts.

Where the biological conduit to be treated is obstructed, the obstruction can be, for example, a stenosis, stricture, or lesion.

In practicing the methods of the invention described herein, reference can be made to U.S. application Ser. No. 09/669,051 by inventor Franano, filed Sep. 24, 2000, and WO 2004/073504 by inventors Franano and Romano, published on Sep. 2, 2004, the entire contents of which are incorporated by reference herein in their entireties.

4.1. Elastase(s) and/or Collagenases

The present invention provides methods of treating or preventing vasospasm in a biological conduit, and for reducing the accumulation of intimal hyperplasia in a biological conduit after a vascular procedure, comprising, in certain embodiments, administering to the wall of the conduit an elastase and/or collagenase.

### 4.1.1. Collagenases

Collagen is a majority component of the extracellular matrix of multicellular eukaryotic organisms. It is a structural protein which is characterized by regions of small, repeating sequences of amino acids which result in the formation of helical chains between molecules. These helices give rise to its exceptional structural stability and strength. Collagen is the main constituent of the skin, tendons, bones, cartilages and tissues and represents approximately 40% of all the proteins of the human body. Although the collagen molecule is very resistant to the action of most proteases, it can still be degraded by specific proteases referred to as collagenases.

Several members of the enzyme family known as metalloproteinases (MMPs) are collagenases. These enzymes are very widely distributed in the living world and are present, but weakly expressed, in normal physiological situations, such as organ growth and tissue replacement. Their overexpression in man and their activation are related, however, to numerous processes, sometimes pathological processes, which involve the uncontrolled destruction, and the remodelling of extracellular matrix. Two classes of collagenases have been identified and are characterized by the specificity of the cleavage they bring about in the collagen molecule. The first class of collagenases is constituted by collagenases of higher organisms, which hydrolyze the peptide bonds containing Gly—Ile or Gly—Leu, whereas the second class is constituted by bacterial collagenases, which systematically hydrolyze all the peptide bonds having the sequence X—Gly and generally degrade any collagen molecule.

Some enzymes, such as MMP-2, MMP-9, and leukocyte elastase degrade both elastin and some collagens. An agent that degrades elastin rapidly and collagens slowly provides greater dilation than an agent that degrades elastin alone, because of partial collagen degradation and subsequent remodeling after treatment. An agent that degrades collagens but not elastin may be administered directly into the wall of a biological conduit that is obstructed by a collagen-rich tissue, such as intimal hyperplasia, effectively clearing the obstructing material from the lumen of the conduit.

In a preferred embodiment, a collagenase for use in accordance with the present methods and compositions is one that degrades type IV basement membrane collagen.

In alternative embodiments, a collagenase for use in accordance with the present methods and compositions is one that degrades collagens types I, II and III (e.g., matrix metalloprotease types 1, 3, 7, 9 and 10).

In a certain specific embodiment, the collagenase is Clostridium histolyticum collagenase.

### 4.1.2. Elastases

Many enzymes cleave elastin and can, therefore, be considered elastases. The selection of a specific enzyme(s) for use as a therapeutic agent is important. Humans synthesize a family of zinc and calcium dependent endopeptidases called matrix metalloproteinases (MMPs) that have the ability to degrade various components of the extracellular matrix, including some that degrade elastin, some that degrade collagen(s) and some that degrade both. Humans synthesize a Type I elastase known as ELA-1, with 89% amino acid homology to Type I porcine pancreatic elastase. Humans also synthesize a Type II and a Type III pancreatic elastase. These elastases are differentiated by their amino acid sequence and substrate specificity. The porcine pancreas produces several elastases, most notably a Type I elastase that rapidly degrades tropoelastin, proteoglycans, and some glycoproteins. Type I porcine pancreatic elastase is not thought to degrade fibrillar collagens or microfibers, and is not thought to activate PAR receptors. Several preparations of porcine pancreatic elastase are available commercially and highly purified preparations are thought to contain Type I elastase almost exclusively.

In the methods and compositions of the invention utilizing an elastase, the elastase enzyme employed is preferably a Type I Elastase that preferentially cleaves peptide substrates with small hydrophobic amino acid residues such as alanine. Examples of Type I elastases include the human elastase I enzyme (NCBI Accession Number NP_{—}001962) that is expressed in skin and the porcine elastase I enzyme (NCBI Accession Number CAA27670) that is expressed in the pancreas. Alternatively, a Type II Elastase that can cleave peptide substrates with medium to large hydrophobic amino acid residues in the P1 position (i.e., the substrate amino acid residue immediately n-terminal to the scissile bond) may be used. Examples of Type II elastases include the human elastase IIA enzyme (NCBI Accession Number NP254275) and the porcine elastase II enzyme (NCBI Accession Number A26823) that are both expressed in the pancreas.

In the present invention, elastin-degrading agents include, but, are not limited to human pancreatic elastase I (also known as ELA-1), human pancreatic elastase IIA, human pancreatic elastase IIB, human pancreatic elastase IIIA, human pancreatic elastase IIIB, porcine pancreatic elastase I, porcine pancreatic elastase II, porcine pancreatic elastase III, pancreatic elastases from other mammals, including mouse, rat, cow, horse, human leukocyte elastase, matrix metalloproteinase-2 (also known as gelatinase A or 72 kd Type IV collagenase), matrix metalloproteinase-9 (also known as gelatinase B or 92 kd Type IV collagenase), matrix metalloproteinase-7 (also known as matrilysin or PUMP-1), matrix metalloproteinase-12 (also known as human macrophage elastase or human macrophage metalloelastase), cathepsin L, and cathepsin S. In a preferred embodiment, the elastin-degrading agent is a human elastin-degrading agent. In other embodiments, the elastin-degrading agent is from other mammals such as mouse, rat, pig, cow, or horse.

4.2. Agents that Increase Local Concentration of Elastase(s) and/or Collagenase(s)

The present invention provides methods of treating or preventing vasospasm in a biological conduit, and for reducing the accumulation of intimal hyperplasia in a biological conduit after a vascular procedure, comprising, in certain embodiments, administering to the wall of the conduit an agent that stimulates the synthesis and/or release of elastases and collagenases by cells that normally reside in the vessel wall.

4.3. Agents that Induce Inflammation

### 4.3.1. Chemotactic Agents

Another aspect of the present invention provides methods of treating or preventing vasospasm in a biological conduit, and for reducing the accumulation of intimal hyperplasia in a biological conduit after a vascular procedure, comprising, in certain embodiments, administering to the wall of the conduit an agent that that results in the recruitment of monocytes, macrophages, and/or polymorphonuclear (PMN) cells capable of synthesizing and releasing elastases and collagenases in the conduit wall. In some embodiments, the administered agent would be chemotactic for these cells.

### 4.3.2. Inducing the Local Production of Chemotactic Factors

In other embodiments, the agent would cause the local synthesis and/or release of endogenous agents that are chemotactic for monocytes, macrophages, or PMNs. In some embodiments, the administered agent can activate one or more members of the G-protein coupled proteinase activated receptor (PAR) family. Four distinct PARs are known, and they have been given the names PAR-1 (thrombin receptor), PAR-2, PAR-3, and PAR-4. PARs are activated when an n-terminal peptide is cleaved from the receptor, revealing a tethered ligand that inserts into the receptor-binding site. PAR receptor activation often leads to tissue inflammation and the recruitment of monocytes, macrophages, and PMNs. In some embodiments, the agent causes an increase in the endothelial cell surface expression of adhesion molecules for monocytes, macrophages, and/or PMNs, including intercellular adhesion molecules (ICAMs), vascular cell adhesion molecules (VCAMs), and selectins. In other embodiments, the agent causes increased expression of endogenous PAR receptors in the target tissue. Preferably, the administered agent is selected from pancreatic elastase, trypsin, trypsin iv, mesotrypsin1, chymotrypsin, mast cell tryptase, neutrophil proteinase-1, tissue factor, factor VIIa, factor Xa, thrombin, plasmin, cathepsin G, MCP-1, synthetic peptides which activate PARs, peptidomimetic or other small-molecule PAR agonists, macrophage elastase, leukocyte elastase, and all members of the family of proteases known as matrix metalloproteinases (Cottrell et al., 2004, J. Biol. Chem. 2004 Jan. 15 [Epub ahead of print]). Alternatively, agents that induce expression of endogenous PAR-2 such as TNF-alpha, IL-1 or bacterial Lipopolysaccharide (LPS) are used (Nystedt et al., J. Biol. Chem. 271:14910).

### 4.4. Agents That Degrade Microfibrils

Another aspect of the present invention involves the administration of an agent that degrades microfibers and/or fibrillins to an agent that degrades tropoelastin, in the practice of the methods of the invention. Preferably, the administered agent is selected from trypsin, chymotrypsin, and plasmin, and all members of the family of proteases known as matrix metalloproteinases.

In the present invention, microfiber degrading agents include, but, are not limited to human trypsin, trypsin from other mammals including mouse, rat, pig, cow, horse, human chymotrypsin, chymotrypsin from other mammals including mouse, rat, pig, cow, horse, human plasmin, plasmin from other mammals including mouse, rat, pig, cow, horse, human leukocyte elastase, leukocyte elastase from other mammals including mouse, rat, pig, cow, horse,

In various embodiments of the present invention, the microfiber-degrading agent is matrix metalloproteinase-2 (also known as gelatinase A or 72 kd Type IV collagenase), matrix metalloproteinase-9 (also known as gelatinase B or 92 kd Type IV collagenase), matrix metalloproteinase-7 (also known as matrilysin or PUMP-1), or matrix metalloproteinase-12 (also known as human macrophage elastase or human macrophage metalloelastase). In a preferred embodiment, the matrix metalloproteinase is a human matrix metalloproteinase. In other embodiments, the matrix metalloproteinase is from other mammals such as mouse, rat, pig, cow, or horse.

### 4.5. Combination Therapy

Described below are combination methods and related compositions for treating or preventing vasospasm in a biological conduit, and for reducing the accumulation of intimal hyperplasia in a biological conduit after a vascular procedure, comprising, in certain embodiments, administering to the wall of the conduit an agent that. The methods of the invention involve the administration of at least two agents to a patient, the first of which has diameter-enlarging activity, either directly or indirectly. The second agent is generally capable of enhancing the effect of the first agent, either through facilitating the delivery of the first agent, or through exerting direct (e.g., by degrading elastin) or indirect (e.g., by inducing local inflammation of the conduit) diameter-enlarging activity. In certain embodiments, the combination methods further encompass administering a third agent that is generally capable of enhancing the effect of the first or second agent, either through facilitating the delivery of the first or second agent, or through exerting direct or indirect diameter-enlarging activity. These agents are administered, however, in dosages that do not result in dilation of the biological conduits.

Accordingly, in certain embodiments, the methods of the invention encompass the combination administration of a combination of any (e.g., two, three, four, five, six or all) of the following types of agents: (1) an elastase; (2) a collagenase; (3) an agent that increases the local concentration of one or more endogenous elastases or collagenases; (4) an agent that induces local inflammation in the segment of the conduit to which it is administered; (5) an agent that degrades microfibers in the wall of the segment of the conduit to which it is administered; (6) a chemotactic factor for monocytes, macrophages, or polymorphonuclear cells; (7) a macrophage-activating agent; and (8) an agent that degrades proteoglycans and/or glycoproteins.

In preferred embodiments of the combination methods disclosed herein, the combination methods comprise the administration of an elastase or a collagenase and at least one of the agents listed in (3)-(8) above that is not an elastase or a collagenase.

In other preferred embodiments of the methods disclosed herein involving the administration of an elastase or collagenase, the elastase or collagenase does not display any one, two, three or four, or all five, of the following activities: (a) increasing the local concentration of one or more endogenous elastases or collagenases; (b) inducing local inflammation; (c) degrading microfibers; (d) increasing the local concentration of an endogenous chemotactic factor for monocytes, macrophages, or polymorphonuclear cells; (e) activating macrophages; (f) degrading extracellular matrix in the conduit; and/or (g) degrading proteoglycans or glycoproteins in the wall of the conduit. These agents are administered, however, in dosages that do not result in dilation of the biological conduits.

Preferably, where the combination methods comprise the administration of a chemotactic factor for monocytes, macrophages, or polymorphonuclear cells, a macrophage-activating agent is also administered.

Further, the combination methods of the invention encompass performing a combination of any (e.g., two, three, four, five, six or all) of the following methods: (1) an administering an elastase; (2) administering a collagenase; (3) increasing the local concentration of one or more endogenous elastases or collagenases; (4) inducing local inflammation in the segment of the conduit to be treated; (5) degrading microfibers in the wall of the segment of the conduit to be treated; (6) increasing the local concentration of an endogenous or exogenous chemotactic factor for monocytes, macrophages, or polymorphonuclear cells; (7) activating macrophages in the segment of the conduit to be treated; (8) degrading extracellular matrix in the conduit; and/or (9) degrading proteoglycans or glycoproteins in the wall of the conduit. These agents are administered, however, in dosages that do not result in dilation of the biological conduits.

The combination therapy methods of the present invention often result in a synergistic effect, i.e., a greater than additive effect that would be expected from the agents separately. In some instances, the combination therapy methods of the present invention provide therapeutic benefits where neither agent utilized in combination therapy is effective in isolation. The greater than additive effects can be achieved, for example where the first agent is administered in an amount that is sub-therapeutic. In other instances, the combination therapy methods of the present invention provide benefits greater than the sum of administering each agent alone.

In the present methods, the first agents and second agent can be administered concurrently or successively. As used herein, the agents are said to be administered concurrently if they are administered to the patient on the same day, for example, simultaneously, or 1, 2, 3, 4, 5, 6, 7 or 8 hours apart. In contrast, the agents are said to be administered successively if they are administered to the patient on the different days, for example, the first and second agent can be administered at a 1 day, 2-day or 3-day intervals.

### 4.6. Effective Dose

The present invention generally provides the benefit of parenteral, preferably local, administration of agents for treating or preventing disease in biological conduits.

In certain embodiments, as an alternative to parenteral administration, or, where a combination therapy method is utilized, in addition to parenteral administration, oral administration of agents for treating or preventing disease in biological conduits may be used.

Toxicity and therapeutic efficacy of the agents utilized in the practice of the methods of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Such information can be used to more accurately determine useful doses in humans.

In certain embodiments, the dosage of administered, e.g., elastase or collagenase, is at least about 10% less than the dose that would be effective to achieve a dilation of a biological conduit. In other embodiments, the dosage is at least about 30%, 40%, 50%, 60%, 70%, 80%, or 90% less than the dose that would be effective to achieve a dilation of a biological conduit. The dose that would be effective to achieve a dilation of a biological conduit can be measured as described in U.S. application Ser. No. 09/669,051 by inventor Franano, filed Sep. 24, 2000, or in WO 2004/073504 by inventors Franano and Romano, published on Sep. 2, 2004, the entire contents of which are incorporated by reference herein in their entireties.

In embodiments utilizing an elastase as the sole agent employed in the methods of the invention, the dosage of elastase is 1 to 10 units, 10-25 units, 25-50 units, 50-100 units, or 100-500 units. Elastase units are defined as μmole of substrate hydrolyzed per mg per minute at pH 8.0 and 25 oC in 0.1 M Tris pH 8.0 with 0.5 mM Succinyl-alanine-alanine-alanine-pNA, read at 410 nm. Specific activity is 12 U/mg or highly purified Type I porcine pancreatic elastase, using this assay.

### 4.7. Formulations and Methods of Administration

The invention relates to pharmaceutical compositions and methods of use thereof for preventing or treating disease in biological conduits. Such pharmaceutical compositions can be formulated in a conventional manner using one or more physiologically acceptable carriers or excipients.

In embodiments of the present invention encompassing combination therapy with one or more agents, the one or more agents can be formulated into one pharmaceutical composition, most preferably in amounts that are effective to treat or prevent preventing or treating disease in biological conduits. In alternative embodiments, the one or more agents can be formulated into separate pharmaceutical compositions.

Most preferably, in the compositions of the invention comprising one or more agents useful for practicing the methods of the invention (e.g., one or more of: (1) an elastase; (2) a collagenase; (3) an agent that increases the local concentration of one or more endogenous elastases or collagenases upon its administration to a biological conduit; (4) an agent that induces local inflammation upon its administration to a biological conduit; (5) an agent that degrades microfibers upon its administration to a biological conduit; (6) an agent that increases the local concentration of an endogenous or exogenous chemotactic factor for monocytes, macrophages, or polymorphonuclear cells upon its administration to a biological conduit; (7) an agent that activates macrophages; (8) an agent that degrades extracellular matrix upon its administration to a biological conduit; and/or (9) an agent that degrades proteoglycans or glycoproteins upon its administration to a biological conduit), at least one or more agents are purified to a pharmaceutical grade prior to their formulation into a composition of the invention. In certain specific embodiments, the degree of purity of at least one or more agents prior to such formulation is such that there is no detectable enzymatic activity of any of the other agents suitable for practicing the methods of the invention. Thus, in certain preferred embodiments of the invention, a composition to be administered in accordance with the methods of the invention is prepared by combining a first purified enzyme, e.g., an elastase, in combination with a second purified enzyme, e.g., trypsin.

The agents utilized in the methods of the present invention are generally administered parenterally, often directly to the segment of the biological conduit being treated. Formulations for parenteral administration can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

Where oral administration is desired, for example for administering PAR antagonists, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active agent.

The agents of the present invention can be administered to the desired segment of the biological conduit being treated by any device known to one of skill in the art to be cardiovascular delivery, e.g., a syringe, a drug delivery catheter, an implanted drug delivery polymer, such as a sheet or microsphere preparation, an implantable venous catheter, a venous port, a tunneled venous catheter, a chronic infusion line or port, or a polymer-coated vascular stent, preferably a self-expanding stent.

In certain embodiments, the administration to the desired segment may be guided by ultrasound, CT, fluoroscopic guidance, MRI or endoscopic guidance.

In certain aspects of the present invention, administration of an agent to a biological conduit comprises localizing a delivery apparatus in close proximity to the segment of the biological conduit to be treated. In some embodiments, during delivery of the agent by a delivery apparatus, a portion of the delivery apparatus can be inserted into the wall of the biological conduit. In some embodiments, the lumen of the biological conduit can be pressurized while the agent is delivered to the pressurized segment of the biological conduit. In some embodiments, the lumen of the biological conduit is pressurized by mechanical action. In some embodiments, the lumen of the biological conduit is pressurized with a balloon catheter. In some embodiments, the agent is administered and the pressurizing is performed by the same device. In some embodiments, the biological conduit is surgically exposed and the agent is delivered into the lumen or is applied to the external surface of the biological conduit in vivo. In embodiments involving luminal delivery, blood flow through the vessel may be stopped with a clamp to allow the agent to contact the endothelium surface for longer time periods and to prevent inhibition of the agent by serum. In some embodiments, the biological conduit is surgically removed and the agent is delivered to the luminal surface and/or to the external surface of the conduit in vitro.

In other aspects of the present invention, administration of an agent to a biological conduit entails the use of a polymer formulation that is placed as a stent within the vessel to be treated, a clamp or wrap on or around the vessel to be treated, or other device in, around or near the vessel to be treated.

In yet other aspects of the present invention, agents are percutaneously injected into a tissue region for purpose of dilating arteries and/or vein within that region, including collateral arteries. In embodiments aimed at treatment of heart vessels, agents are either percutaneously delivered to the pericardial space or directly applied to surgically exposed coronary vessels.

### 4.8. KITS

The present invention provides kits for practicing the methods of the present invention. A kit of the invention comprises in one or more containers one or more of the agents described herein as useful for treating or preventing disease in biological conduits in the dosages described herein.

The kit of the invention may optionally comprise additional components useful for performing the methods of the invention. By way of example, the kit may comprise pharmaceutical carriers useful for formulating the agents of the invention. The kit may also comprise a device or a component of a device for performing the methods of the invention, for example a syringe or needle. In addition or in the alternative, the kits of the invention may provide an instructional material which describes performance of one or more methods of the invention, or a notice in the form prescribed by a governmental agency regulating the manufacture, use or sale of pharmaceuticals or biological products, which notice reflects approval by the agency of manufacture, use or sale for human administration.

### SPECIFIC EMBODIMENTS, CITATION OF REFERENCES

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references, including patent applications, patents, and scientific publications, are cited herein; the disclosure of each such reference is hereby incorporated herein by reference in its entirety.

What is claimed is:

1. A method of reducing vasospasm in a segment of a biological conduit, said method comprising:
administering to said segment in a human subject in need thereof, via a parenteral route, a composition comprising an elastase,
wherein the amount of elastase administered is insufficient to cause dilation of the treated segment, but sufficient to reduce vasospasm of at least a segment of a biological conduit.

2. A method of reducing the ability of a vessel to undergo vasospasm in a segment of a biological conduit, said method comprising:
administering to said segment in a human subject in need thereof, via a parenteral route, a composition comprising an elastase,
wherein the amount of elastase administered is insufficient to cause dilation of the treated segment, but sufficient to reduce the ability of at least a segment of a biological conduit to undergo vasospasm.

3. A method of reducing the accumulation of intimal hyperplasia in a segment of a biological conduit, said method comprising:
administering to said segment in a human subject in need thereof, via a parenteral route, a composition comprising an elastase,
in an amount insufficient to cause dilation of the treated segment, but in amount sufficient to
wherein the amount of elastase administered is insufficient to cause dilation of the treated segment, but sufficient to reduce the accumulation of intimal hyperplasia in at least a segment of a biological conduit.

4. The method of any one of claims 1-3, wherein the biological conduit is an artery or vein, or an arterial or venous vascular graft.

5. The method of any one of claims 1-3, wherein said composition is administered directly to said biological conduit.

6. The method of claim 5, wherein said composition is administered by a catheter.

7. The method of claim 5, wherein said composition is administered to a surgically exposed segment of the biological conduit within the human subject.

8. The method of claim 5, wherein the composition is delivered into the lumen of the biological conduit.

9. The method of claim 5, wherein the composition is applied to the external surface of the biological conduit.

10. The method of any one of claims 1-3, wherein said composition is administered percutaneously into a tissue comprising said biological conduit.

11. The method of any one of the claims 1-3, wherein the biological conduit is a coronary artery or a vein bypass graft connected to a coronary artery.

12. The method of any one of the claims 1-3, wherein the composition is administered percutaneously into the pericardial space.

13. The method of claim 1 or 2, wherein the vasospasm is reduced by 1% to 5%, by 5% to 25%, by 25% to 50%, or by 50% to 100%.

14. The method of claim 1 or 2, wherein the vasospasm in response to mechanical or pharmacologic stimuli is reduced by 1% to 5%, by 5% to 25%, by 25% to 50%, or by 50% to 100%, compared with untreated vessels.

15. The method of claim 3, wherein the accumulation of intimal hyperplasia is reduced by 1% to 5%, by 5% to 25%, by 25% to 50%, or by 50% to 100% at 1 month, 3 months, 6 months, or 1 year after treatment.

16. The method of claims 1-3, wherein the dose of elastase is 1 to 10 units, 10-25 units, 25-50 units, 50-100 units, or 100-500 units.
